(19) United States
(12) Patent Application Publication
(10) Pub. No.: US 2025/0364123 A1
(43) Pub. Date: Nov. 27, 2025

(54) MONITORING OF A MEDICAL ENVIRONMENT BY FUSION OF EGOCENTRIC AND EXOCENTRIC SENSOR DATA

(71) Applicant: Intuitive Surgical Operations, Inc., Sunnyvale, CA (US)

(72) Inventors: Omid Mohareri, San Francisco, CA (US); Muhammad Abdullah Jamal, Sunnyvale, CA (US)

(73) Assignee: Intuitive Surgical Operations, Inc., Sunnyvale, CA (US)

(21) Appl. No.: 19/197,514

(22) Filed: May 2, 2025

Related U.S. Application Data
(60) Provisional application No. 63/650,300, filed on May 21, 2024.

(51) Int. Cl. G16H 40/20; A61B 34/30; G16H 40/40

(52) U.S. Cl. CPC G16H 40/20; A61B 34/30; G16H 40/40

(57) ABSTRACT

Aspects of this technical solution can receive a first set of data from an exocentric sensor, the exocentric sensor being configured to capture information of a medical environment, receive a second set of data from an egocentric sensor, the egocentric sensor being configured to capture egocentric information from a perspective of a first medical personnel in the medical environment, receive a third set of data from a computer-assisted medical system, and generate, using one or more machine-learning models, a set of procedure information for a medical procedure performed in the medical environment based on the first set of data from the exocentric sensor, the second set of data from the egocentric sensor, and the third set of data from the computer-assisted medical system.

### CROSS-REFERENCE TO RELATED APPLICATION

This application claims the benefit of, and priority to, U.S. Patent Application No. 63/650,300, filed May 21, 2024, the full disclosure of which is incorporated herein in its entirety.

### TECHNICAL FIELD

The present implementations relate generally to medical equipment, including but not limited to monitoring of a medical environment by fusion of egocentric and exocentric sensor data.

### INTRODUCTION

Patient care is becoming increasingly complex and involves increasingly specialized medical staff in increasing numbers. The introduction of computerization and computerized medical technologies has further increased the complexity of properly maintaining and executing sound processes in clinical environments. Conventional systems cannot effectively or efficiently maintain holistic or up-to-date awareness of medical environments at levels of accuracy expected in medical contexts.

### SUMMARY

Systems, methods, apparatuses, and non-transitory computer-readable media are provided for generating a plurality of types of metrics descriptive of a medical procedure or medical environment, based on the fusion of one or more first sensors positioned with respect to the medical environment and one or more second sensors positioned with respect to individuals in the medical environment. For example, the first sensors can include one or more exocentric sensors configured to capture a third person view (TPV) of the medical environment (e.g., fixed on a wall or ceiling of a medical environment, and second sensors can include one or more egocentric sensors configured to capture corresponding first person views (FPVs) of the medical environment. According to some embodiments, an egocentric sensor can include a wearable sensor worn by or affixed to medical staff personnel. The exocentric sensors and the egocentric sensors can capture multi-modal data (e.g., depth data and visual image data) of the medical environment from their respective viewpoints. For example, the exocentric sensors can provide a TPV from a corner, ceiling, or wall of the medical environment (e.g., an operating room (OR)), and the wearable sensors can provide an FPV from respective body-worn cameras aligned with a field of view of the medical personnel. Thus, the exocentric sensors can each provide a distinct “exocentric” view independent of the pose (e.g., position and orientation) of any specific individual in the medical environment, and the egocentric sensors can each provide a distinct “egocentric” view that corresponds to a viewpoint of a specific individual in the medical environment throughout a medical procedure performed in the medical environment.

Based on sensor data generated by the exocentric and egocentric sensors, a system can generate metrics descriptive a given medical procedure performed in the medical environment. Combining visual and depth data from multiple sensors with artificial intelligence (AI) or machine learning (ML) models can significantly improve the capture of data concerning the locations and movements of humans and objects throughout a physical environment (e.g., the medical environment) in which probability of occlusion of a single sensor positioned in the medical environment (whether at a fixed posed or a dynamic pose) can be high. Because of the high granularity and narrow margin of error for various tasks of a medical procedure, the arrangements disclosed herein can improve robotically-assisted medical systems. For example, it can be recognized that a medical procedure in which a robotically-assisted medical system such as a robotic surgery system is deployed can have a greater number of occlusion issues as compared to a non-robotic medical procedure due to the various types of frequent interactions between the medical personnel/patient with the robotic surgery system, where such interactions would not exist and would not be occluded by parts of the robotic surgery system in non-robotic medical procedures. Accordingly in some embodiments, the system can include a spatial registration method to synchronize multi-modal data from one or more sensors of egocentric and exocentric sensor types into one coordinate frame (e.g., a common coordinate frame or system) and timeline and provide a representation of the medical environment from multiple static and dynamic fields of view to minimize occlusion and maximize visibility of the entire medical procedure or medical environment with responsiveness and accuracy beyond the capability of manual processes to achieve.

In some embodiments, data obtained from at least one egocentric sensor and at least one exocentric sensor can be time-synchronized, spatially registered and integrated by one or more systems or devices as discussed herein. Time-synchronization can include aligning one or more timestamps corresponding to data (e.g., depth data, image data, or video data) generated or captured by at least one exocentric sensor, with one or more timestamps corresponding to data (e.g., depth data, image data, or video data) generated or captured by at least one egocentric sensor. For example, timestamps can be aligned according to detection of various objects or persons in one or more frames, and assigning a common time or a plurality of sensors or an offset from a common time or a time associated with one of the sensors (e.g., an exocentric sensor associated with a camera).

Spatial registration can correspond to applying a common coordinate frame to one to more sensors, to provide a common frame of reference (e.g., the common coordinate system) for locations, positions, and movements of objects and persons within the medical environment. In some embodiments, exocentric sensors are located in a fixed location in the medical environment and have a specific field of view. Based on this information, a specific 3D volume can be defined to represent the area in the medical environment that's covered by one or more exocentric sensor (V_{TPV(i)}). In some embodiments, one or more egocentric sensors are placed on or near a body (e.g., a head, a chest, a hand, etc.) of a person and have corresponding fields of views (V_{FPV(j)}). For example, a field of view can correspond to a volume that can be captured by the sensor, and a field of view can correspond to a portion of a scene that can be captured without occlusion in the scene (e.g., occlusion by objects or persons).

In some embodiments, because exocentric sensors and egocentric sensors are spatially and temporally registered, the overlap between coverage volumes between each pair or sensors (e.g., one ego centric sensor and one exocentric sensor, two egocentric sensors, or two exocentric sensors) can be computed in real time, e.g., (V_{FPV-TPV(i,j)}) to identify locations, positions, and movements of objects and persons within a common coordinate frame of the medical environment. Thus, data from one or more egocentric sensors can be combined with data from one or more exocentric sensors for enhancing each individual sensor data and help with resolving occlusions. In some embodiments, even though occlusion may occur with respect to a portion of an object or person at an exocentric sensor or an egocentric sensor, the system can identify sensor data available from another egocentric sensor or exocentric sensor that cover the same 3D volume in the medical environment, and provide additional data including the data from the other sensors to reduce or eliminate the occlusion.

In some embodiments, each egocentric sensor has a view direction defined by a corresponding vector (T_{FPV(i)}) for a field of view of the egocentric sensor that corresponds to a direction or orientation in which the egocentric sensor is facing and aimed to collect data. This vector can be represented in the medical environment world coordinate frame (e.g., the common coordinate frame), to which exocentric sensors are also registered. Therefore, object detection, activities recognition, and other methods described herein can be performed with respect to data registered to the common coordinate frame. For example, a system recognizes that a team is performing a “port placement” activity in a medical environment. The system recognizes that five people are present during this task, for example, in the medical environment or within a portion of the medical environment within a predetermined distance from a task site or a predetermined volume linked with the task site. The system recognizes that two people are working on “port placement” together while others are performing other unrelated tasks, or are not performing port placement.

At least one aspect is directed to a system. The system can include one or more processors coupled with memory. The system can receive exocentric data from an exocentric sensor having a first pose in a medical environment, the exocentric data capturing the medical environment from the first pose, the first pose being stationary within the medical environment. The system can receive egocentric data from an egocentric sensor having a second pose in the medical environment, the egocentric data capturing the medical environment from the second pose, where the second pose is dynamic with respect to the medical environment, and the second pose is configured to change according to the movement of a user. The system can determine, based at least in part on the exocentric data and the egocentric data, a timeline that can include at least one phase identified for a medical procedure within the medical environment and at least one task identified within the at least one phase. The system can determine a metric for the at least one task or the at least one phase.

### BRIEF DESCRIPTION OF THE FIGURES

These and other aspects and features of the present implementations are depicted by way of example in the figures discussed herein. Present implementations can be directed to, but are not limited to, examples depicted in the figures discussed herein. Thus, this disclosure is not limited to any figure or portion thereof depicted or referenced herein, or any aspect described herein with respect to any figures depicted or referenced herein.

FIG. 1A depicts an example architecture of a system according to this disclosure.

FIG. 1B depicts an example environment of a system according to this disclosure.

FIG. 2 depicts an example sensor control system according to this disclosure.

FIG. 3 depicts an example layer model architecture according to this disclosure.

FIG. 4A depicts an example first state of a medical environment according to this disclosure.

FIG. 4B depicts an example second state of a medical environment according to this disclosure.

FIG. 5 depicts an example medical environment in an interaction state according to this disclosure.

FIG. 6 depicts an example medical environment in a social state according to this disclosure.

FIG. 7 depicts an example first-person view device according to this disclosure.

FIG. 8 depicts an example augmented reality presentation according to this disclosure.

FIG. 9 depicts an example method of identifying procedural states of an environment by fusion of egocentric and exocentric sensor data, according to this disclosure.

FIG. 10 depicts an example method of identifying procedural states of an environment by fusion of egocentric and exocentric sensor data, according to this disclosure.

FIG. 11 depicts an example method of monitoring of a medical environment by fusion of egocentric and exocentric sensor data, according to this disclosure.

### DETAILED DESCRIPTION

Aspects of this technical solution are described herein with reference to the figures, which are illustrative examples of this technical solution. The figures and examples below are not meant to limit the scope of this technical solution to the present implementations or to a single implementation, and other implementations in accordance with present implementations are possible, for example, by way of interchange of some or all of the described or illustrated elements. Where certain elements of the present implementations can be partially or fully implemented using known components, only those portions of such known components that are necessary for an understanding of the present implementations are described, and detailed descriptions of other portions of such known components are omitted to not obscure the present implementations. Terms in the specification and claims are to be ascribed no uncommon or special meaning unless explicitly set forth herein. Further, this technical solution and the present implementations encompass present and future known equivalents to the known components referred to herein by way of description, illustration, or example.

Systems, methods, apparatuses, and non-transitory computer-readable media are provided for identifying procedural states of an environment by fusion of egocentric and exocentric sensor data. For example, each of a plurality of exocentric sensors and wearable sensors can capture depth data and image data of the medical environment from viewpoints within the medical environment. For example, the medical environment sensors can provide a TPV from at least one exocentric sensor (e.g., a camera mounted to a corner or wall of the medical environment), and the wearable sensors can provide a FPV from respective egocentric sensors (e.g., head-worn cameras aligned with a field of view of various medical environment staff). Thus, the medical environment sensors can each provide a distinct “exocentric” view independent of any individual in the medical environment, and the wearable sensors can each provide a distinct “egocentric” view that corresponds to a viewpoint of a specific individual in the medical environment throughout the medical procedure. Based on sensor input from exocentric and egocentric sensors, a system can generate multiple metrics and multiple types of metrics descriptive of the medical environment and/or a medical procedure performed in the medical environment. Thus, a technical solution for generating information (e.g., procedure information, individual information) related to a medical procedure through the fusion of egocentric and exocentric sensor data is provided.

In some embodiments, a system can generate one or more of memory metrics, interaction metrics, and social metrics based on fusion of input from various exocentric and egocentric sensors. For example, the system can generate one or more of these metrics substantially in real time during a medical procedure. Memory metrics can be indicative of states of given individuals or objects at given times. For example, based on egocentric and exocentric data, a system can track the location of an object from place to place as it is handled by one or more individuals. For example, the system can remind a user where a specific instrument was placed by that user or another individual in the environment, if the user has completed a given task (e.g., has cleaned all equipment), or can recall one or more activities performed by the user. Thus a system can concurrently track state of an environment and multiple objects in the environment, and share those states in real-time between one or more (e.g., all) of the personnel in the medical environment. For example, the system can remind a first person where they placed an object, or they can inform a second person that the first person placed an object in a certain location. Interaction metrics can be indicative of locations, movements, or actions of one or more individuals or objects during a medical procedure. For example, the system can leverage one or more egocentric sensors to accurately identify when, where and how an object is changed during its interaction. Social metrics can be indicative of relationships between individuals and objects in a medical environment during a medical procedure. For example, the system can identify one or more individual or objects performing a given task of a medical procedure, based on image recognition of movements and locations of the individuals with respect to each other, one or more objects (e.g., medical instruments) in the medical environment, or any combination thereof. For example, the system can capture utterances and nonverbal cues from each participant's unique view to determine or classify various arrangements and movements of individual and objects collectively as indicative of various tasks or social interactions associated with the medical procedure or the medical environment. The system can track a plurality of objects at a level of accuracy that exceeds the capability of manual processes.

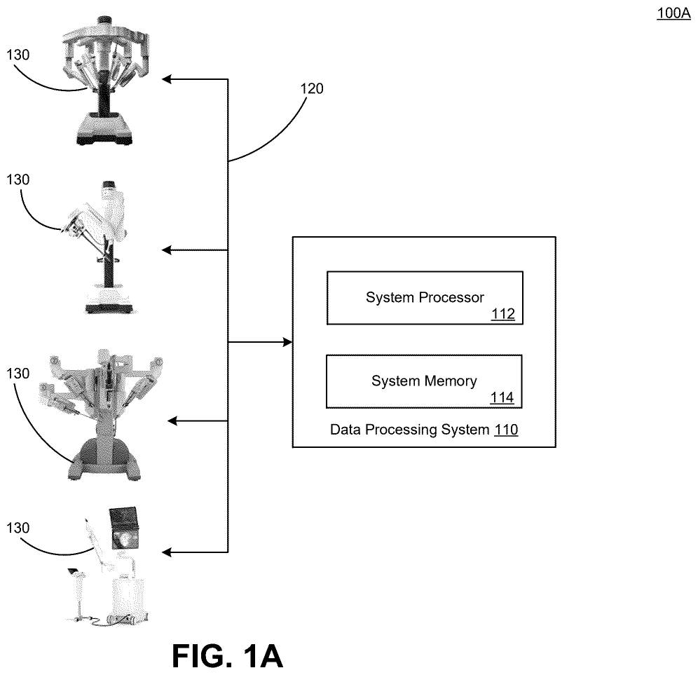

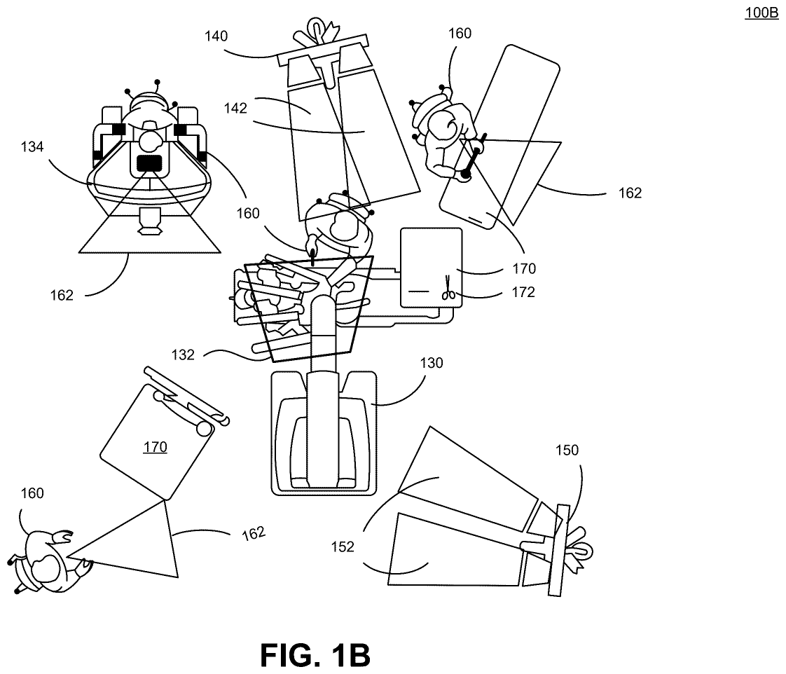

FIG. 1A depicts an example architecture of a system according to this disclosure. As illustrated by way of example in FIG. 1A, an architecture of a system 100A can include at least a data processing system 110, a communication bus 120, and a robotic manipulator system 130. In some embodiments, the system 100A can configure multiple sensors in the medical environment based on the detection of a state or scene corresponding to the medical environment, as a whole. The system can detect, for example, a robot docking scene, as discussed above, and can configure multiple sensors in the medical environment according to the field of view of the sensor or the location of the sensor in the medical environment. For example, the system can enter a training mode in which a model is trained with machine learning from input, including video from a plurality of camera sensors distributed within the medical environment. The machine learning model can optimize the configuration of the robotic manipulator system 130 for a given surgeon during a given medical procedure using a loss function that is based on at least one of the video data, parameters assigned to the surgeon, positions assigned to body parts of the surgeon at the surgeon console, or any combination thereof.

The machine learning model can treat video input from each of these sensors as a combined input for determining optimized allocation or a loss. This way, the machine learning model can be updated (e.g., trained) to provide a technical improvement to increase accuracy of configuration of a robotic system for the ergonomic state of an individual operator (e.g., a surgeon at a surgeon console of the robotic system), to realize physical configurations of a robotic system responsive to state of a surgeon that varies over time within a medical procedure and across medical procedures. For example, a robotic system can modify a rotational position of a manipulator from a first angular position to a second angular position to counteract, for example, an inward grip twist of a wrist of the surgeon. The robotic manipulator system 130 can execute the modification of the rotation position from the first angular position to the second angular position at a rate below a predetermined threshold. Thus, the robotic manipulator system 130 can accommodate while reducing or eliminating potential disruption to the surgical activity by the surgeon in controlling the manipulators during the medical procedure.

The data processing system 110 can include a physical computer system that is operatively coupled or that can be coupled with one or more components of the system 100A, either directly or indirectly through an intermediate computing device or system. The data processing system 110 can include a virtual computing system, an operating system, and a communication bus to effect communication and processing. The data processing system 110 can include a system processor 112 and a system memory 114.

The system processor 112 can execute one or more instructions associated with the system 112. The system processor 112 can include an electronic processor, an integrated circuit, or the like, including one or more of digital logic, analog logic, digital sensors, analog sensors, communication buses, volatile memory, nonvolatile memory, and the like. The system processor 112 can include, but is not limited to, at least one microcontroller unit (MCU), microprocessor unit (MPU), central processing unit (CPU), graphics processing unit (GPU), physics processing unit (PPU), embedded controller (EC), or the like. The system processor 112 can include a memory operable to store or storing one or more instructions for operating components of the system processor 112 and operating components operably coupled to the system processor 112. The one or more instructions can include at least one of firmware, software, hardware, operating systems, embedded operating systems, and the like. The system processor 112 can include at least one communication bus controller to effect communication between the system processor 112 and the other elements of the system 100A.

The system memory 114 can store data associated with the data processing system 110. The system memory 114 can include one or more hardware memory devices to store binary data, digital data, or the like. The system memory 114 can include one or more electrical components, electronic components, programmable electronic components, reprogrammable electronic components, integrated circuits, semiconductor devices, flip-flops, arithmetic units, or the like. The system memory 114 can include at least one of a non-volatile memory device, a solid-state memory device, a flash memory device, or a NAND memory device. The system memory 114 can include one or more addressable memory regions disposed on one or more physical memory arrays. A physical memory array can include a NAND gate array disposed on, for example, at least one of a particular semiconductor device, integrated circuit device, and printed circuit board device. For example, the system memory 114 can correspond to a non-transitory computer-readable medium as discussed herein. In an aspect, the non-transitory computer-readable medium can include one or more instructions executable by the system processor 112. The processor can generate, via a machine learning model receiving as input the exocentric data and the egocentric data, a metric indicative of a state of at least one person or object in the medical environment during a portion of a workflow of the medical procedure.

The communication bus 120 can communicatively couple the data processing system 110 with the robotic manipulator system 130. The communication bus 120 can communicate one or more instructions, signals, conditions, states, or the like between one or more of the data processing system 110 and components, devices, or blocks operatively coupled or couplable therewith. The communication bus 120 can include one or more digital, analog, or like communication channels, lines, traces, or the like. As an example, the communication bus 120 can include at least one serial or parallel communication line among multiple communication lines of a communication interface. The communication bus 120 can include one or more wireless communication devices, systems, protocols, interfaces, or the like. The communication bus 120 can include one or more logical or electronic devices, including but not limited to integrated circuits, logic gates, flip-flops, gate arrays, programmable gate arrays, and the like. The communication bus 120 can include one or more telecommunication devices, including but not limited to antennas, transceivers, packetizers, and wired interface ports.

The robotic manipulator system 130 can include one or more robotic devices configured to perform one or more actions of a medical procedure (e.g., a surgical procedure). For example, a robotic device can include, but is not limited to, a surgical device that can be manipulated by a robotic device. For example, a surgical device can include, but is not limited to, a scalpel or a cauterizing tool. The robotic manipulator system 130 can include various motors, actuators, or electronic devices whose position or configuration can be modified according to input at one or more robotic interfaces. For example, a robotic interface can include a manipulator with one or more levers, buttons, or grasping controls that can be manipulated by pressure or gestures from one or more hands, arms, fingers, or feet. The robotic manipulator system 130 can include a surgeon console in which the surgeon can be positioned (e.g., standing or seated) to operate the robotic manipulator system 130. However, the robotic manipulator system 130 is not limited to a surgeon console co-located or on-site with the robotic manipulator system 130.

FIG. 1B depicts an example environment of a system according to this disclosure. As illustrated by way of example in FIG. 1B, an environment 100B of a system 100A can include at least the robotic manipulator system 130 having a field of view 132, a surgeon console 134, a first sensor system 140, a second sensor system 150, persons 160, and objects 170 and 172. For example, the environment 100B is illustrated by way of example as a plan view of an medical environment having the robotic manipulator system 130, the first sensor system 140, the second sensor system 150, the persons 160, and the objects 170 and 172 disposed therein or thereabout. The presence, placement, orientation, and configuration, for example, of one or more of the robotic manipulator system 130, the first sensor system 140, the second sensor system 150, the persons 160, and the objects 170 and 172 can correspond to a given medical procedure or given type of medical procedure that is being performed, is to be performed, or can be performed in the medical environment corresponding to the environment 100B. This disclosure is not limited to the presence, placement, orientation, or configuration of the robotic manipulator system 130, the first sensor system 140, the second sensor system 150, the persons 160, the objects 170 and 172, or any other element illustrated herein by way of example. The field of view 132 of the robotic manipulator system 130 can correspond to a physical volume within the environment 100B that is within the range of detection of one or more sensors proximate to, coupled with, or integrated with the robotic manipulator system 130. For example, the field of view 132 can be captured by a sensor as discussed herein (e.g., camera) that is positioned above a surgical site of a patient. For example, the field of view 132 is oriented toward a surgical site of a patient. For example, the field of view 132 can capture a view of the surgical site via the sensor at or proximate to the robotic manipulator system 130 from outside the surgical site (e.g., above the surgical site and framing hands and tools of one or more surgeons and one or more anatomical features being operated on by the one or more surgeons)

The first sensor system 140 can include one or more sensors oriented to a first portion of the environment 100B. For example, the first sensor system 140 can include one or more cameras configured to capture images or video in visual or near-visual spectra and/or one or more depth-acquiring sensors for capturing depth data (e.g., three-dimensional point cloud data). For example, the first sensor system 140 can include a one or more cameras configured to collectively capture images or video. For example, the first sensor system 140 can include a plurality of cameras configured to collectively capture images or video in a panoramic view. The first sensor system 140 can include a field of view 142. The field of view 142 can correspond to a physical volume within the environment 100B that is within the range of detection of one or more sensors of the first sensor system 140. For example, the field of view 142 is oriented toward a surgical site of a patient. For example, the field of view 152 is located behind a surgeon at the surgical site of a patient. In an aspect, the first sensor system 140 can correspond to a vision tower, where the vision tower is a device or component of a robotic system including the vision tower and the robotic manipulator system 130.

The second sensor system 150 can include one or more sensors oriented to a second portion of the environment 100B. For example, the second sensor system 150 can include one or more cameras configured to capture images or video in visual or near-visual spectra and/or one or more depth-acquiring sensors for capturing depth data (e.g., three-dimensional point cloud data). For example, the second sensor system 150 can include a plurality of cameras configured to collectively capture images or video in a stereoscopic view. For example, the second sensor system 150 can include a plurality of cameras configured to collectively capture images or video in a panoramic view. The second sensor system 150 can include a field of view 152. The field of view 152 can correspond to a physical volume within the environment 100B that is within the range of detection of one or more sensors of the second sensor system 150. For example, the field of view 152 is oriented toward the robotic manipulator system 130. For example, the field of view 152 is located adjacent to the robotic manipulator system 130. In an aspect, the second sensor system 150 can correspond to a second vision tower, where the second vision tower is a device or component of a robotic system including the vision tower, the second vision tower and the robotic manipulator system 130.

The persons 160 can include one or more individuals present in the environment 100B. For example, the persons can include, but are not limited to, assisting surgeons, supervising surgeons, specialists, nurses, or any combination thereof. One or more of the persons 160 can be associated with a corresponding personal field of view 162. Each personal field of view 162 within the environment 100B can correspond to a respective physical volume within the environment 100B that is within the range of detection of one or more sensors worn by respective persons 160. For example, the field of view 162 is positioned from a forehead or face of each of the persons 160. For example, the field of view 162 is oriented away from a face of a person to capture a volume corresponding to the line of sight and peripheral vision of the respective person 160. In an aspect, one of the persons 160 can be a surgeon seated at the surgeon console 134. For example, the surgeon console 134 is a device or component of a robotic system including the surgeon console 134, at least one of the vision tower or the second vision tower, and the robotic manipulator system 130. In an aspect, the surgeon console 134 can capture input via one or more human interface devices (e.g., joysticks, buttons, or the like), and can provide control instructions to the robotic manipulator system 130 according to the input.

The objects 170 and 172 can include, but are not limited to, one or more pieces of furniture, instruments, or any combination thereof. For example, the objects 170 and 172 can include tables and surgical instruments.

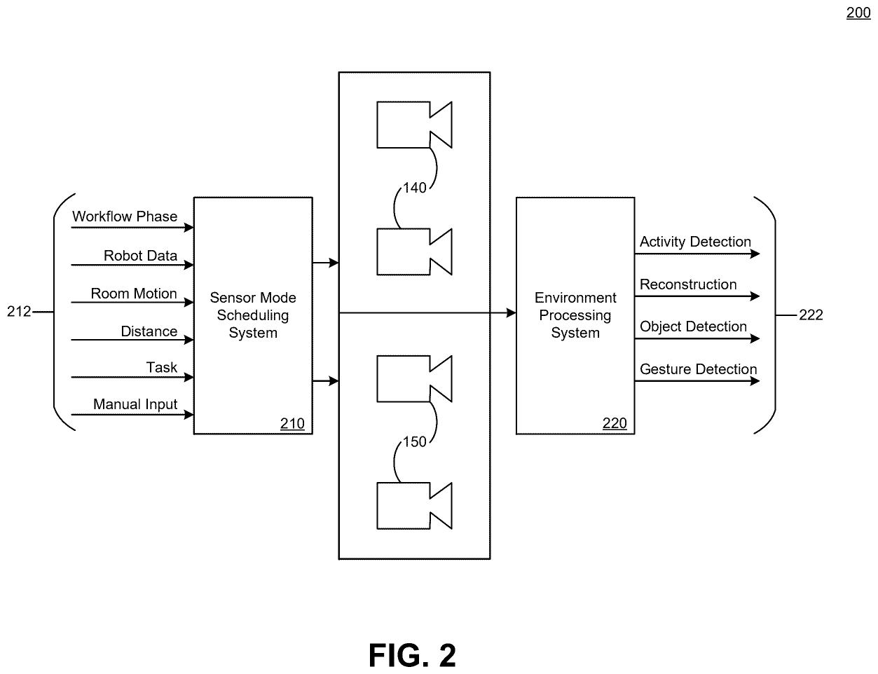

FIG. 2 depicts an example sensor control system according to this disclosure. As illustrated by way of example in FIG. 2, a sensor control system 200 can include at least a sensor mode scheduling system 210, and an environment processing system 220. For example, the sensor control system 200 can be at least partially housed in the data processing system 110, but is not limited thereto. The sensor control system 200 can communicate with the first sensor system 140 and the second sensor system 150 via a wired or wireless communication interface. For example, the communication interface can correspond to or be a component of the communication bus 120.

The sensor mode scheduling system 210 can provide instructions to one or more sensor systems according to or in response to one or more metrics corresponding to the robotic manipulator system 130, the environment 100B, or a medical procedure of the environment 100B, or any combination thereof. For example, the sensor mode scheduling system 210 can include one or more logical or electronic devices including but not limited to integrated circuits, logic gates, flip flops, gate arrays, programmable gate arrays, and the like. One or more electrical, electronic, or like devices, or components associated with the sensor mode scheduling system 210 can also be associated with, integrated with, integrable with, replaced by, supplemented by, complemented by, or the like, the data processing system 110 or any component thereof.

The sensor mode scheduling system 210 can provide instructions to one or more sensors or sensor systems as discussed herein, to change a pose (e.g., a location and/or orientation) or configuration of a given sensor or sensor system as discussed herein, according to one or more input 212. The input 212 can be indicative of a state of the environment 100B, the environment 100B, or the robotic manipulator system 130, or any component, person, or object thereof, any combination thereof. For example, the input 212 can include a workflow phase metric that indicates a current phase of a medical procedure. For example, the input 212 can include a robot data metric that indicates telemetry of one or more components of the robotic device 130. For example, the input 212 can include a room motion metric that indicates aggregate motion of one or more of the persons 160 or objects 170 and 172 in the environment 100B. For example, the input 212 can include one or more distance metrics that each indicate distance traveled by one or more of the persons 160 or objects 170 and 172 in the environment 100B during a given phase. For example, the input 212 can include a task metric that indicates a current task of a medical procedure. For example, the input 212 can include a manual input metric that indicates an instruction for changing a given location, orientation, or configuration of a given sensor or sensor system, as discussed herein. For example, the sensor mode scheduling system 210 can provide instructions to one or more sensors of the first sensory system 140 or the second sensor system 150.

The environment processing system 220 can identify one or more characteristics of the environment 100B. For example, the environment processing system 220 can include a vision architecture, as discussed herein. The environment processing system 220 can generate one or more output metrics 222. For example, the environment processing system 220 can include one or more logical or electronic devices, including but not limited to integrated circuits, logic gates, flip flops, gate arrays, programmable gate arrays, and the like. One or more electrical, electronic, or like devices, or components associated with the environment processing system 220 can also be associated with, integrated with, integrable with, replaced by, supplemented by, complemented by, or the like, the data processing system 110 or any component thereof.

The output metrics 222 can be indicative of a state of a medical procedure of the environment 100B, or any component, person, or object thereof, or any combination thereof. For example, the output metrics 222 can include an activity detection metric that indicates an action being performed by one or more persons in the environment 100B. For example, the activity detection metric can indicate that a person 160, corresponding to a surgeon, is seated at the robotic device 130 and is performing a surgical task. For example, the output metrics 222 can include a reconstruction output that indicates a structure of at least a portion of the environment 100B. For example, the reconstruction output can include a three-dimensional model of at least a portion of the environment 100B during the medical procedure. For example, the output metrics 222 can include an object detection metric that indicates a state of one or more objects in the environment 100B. For example, the object detection metric can indicate that a first object 172, corresponding to a medical instrument (e.g., forceps), is located on a second object 170 corresponding to a table. For example, the environment processing system 220 can first identify one or more objects and can subsequently identify corresponding states for one or more of the identified objects via one or more of the object detection metrics. For example, the output metrics 222 can include a gesture detection metric that indicates a state of one or more body parts of one or more persons 160 in the environment 100B. For example, the gesture metric can indicate that a person 160, corresponding to a surgeon, is holding one or more manipulators of the robotic device 130 by one or more fingers or hands. The embodiments described herein as applied to the system 200 can improve detection of the person 160, the objects 170 and 172, the states of the objects, the surgical task, and so on.

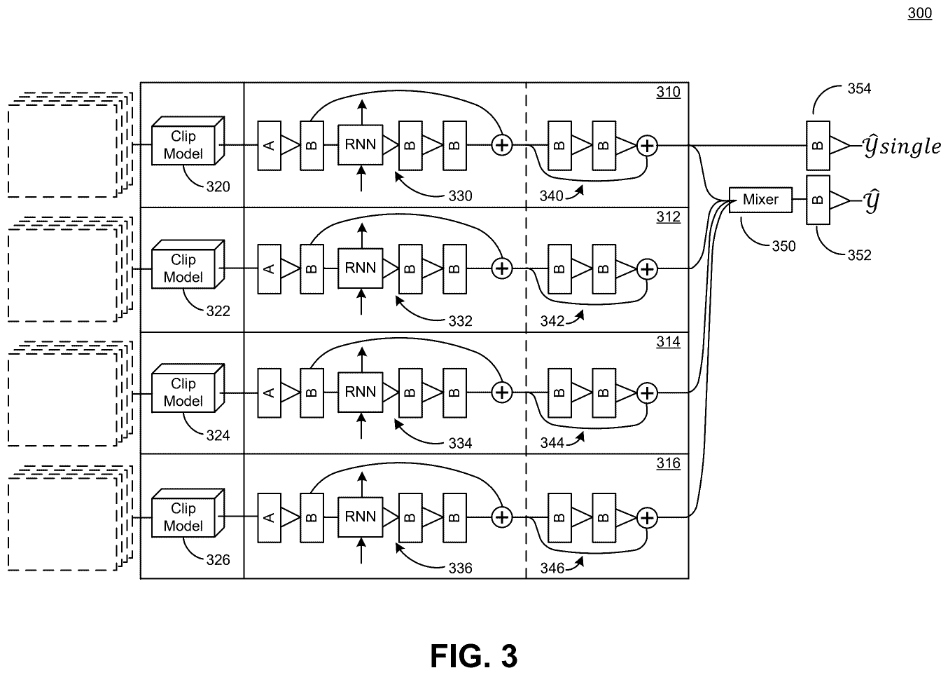

FIG. 3 depicts an example layer model architecture according to this disclosure. As illustrated by way of example in FIG. 3, a layer model architecture 300 can include at least a first layer 310, a second layer 312, a third layer 314, a fourth layer 316, and a mixer 350. The layer model architecture 300 can generate one or more of the features of the environment processing system 220, as discussed herein. For example, the layer model architecture 300 can generate one or more image features as discussed herein, one or more non-image features as discussed herein, or any combination thereof. In an aspect, non-image features can correspond to features directed to aspects of the medical procedure or medical environment other than visual recognition of the medical procedure or the medical environment, or any portion thereof. For example, non-image features can correspond to features directed to workflow phase, robot data, room motion, distance, task, or manual input, but are not limited thereto. For example, the layer model architecture 300 can fuse one or more image features, one or more non-image features, one or more image features with one or more non-image features, or any combination thereof. For example, the environment processing system 220 can include the layer model architecture 300.

The first layer 310 can correspond to the first portion of the environment processing system 220 as discussed herein. The first layer 310 can include a first clip model 320, a first layer processor 330, and a first feature processor 340, and can provide output to a layer output 354. The first clip model 320 can include one or more instructions to receive a video divided into one or more frames and to identify one or more timestamps or times of capture associated with those one or more frames. Such video can refer to a depth video, a visual or color video (e.g., in RGB), and so on captured by an egocentric censor or an exocentric sensor. In some examples, each of the egocentric censor or exocentric sensor can output a stream of videos with suitable timestamps identifying frames. The first layer processor 330 can include a first recurrent neural network (RNN) to identify one or more image features or non-image features as input to the first feature processor 340. The RNN 330 can be coupled with one or more processing devices at inputs and outputs thereof. For example, the processing devices can have different memory capacities, including as illustrated in FIG. 3 by way of example, a first memory size A and a second memory size B. For example, a first memory size “A” can correspond to a memory capacity of 1024 bits or bytes, and a second memory size “B” can correspond to a memory capacity of 128 bits or bytes. However, this disclosure is not limited to the memory sizes or the particular configuration of memory sies illustrated herein by way of example. For example, the RNN 330 can have an input coupled with a first memory device of size A in series with a second memory device of size B, and can have an output coupled with a first memory device of size B in series with a second memory device also of size B. The first feature processor 340 can generate one or more of the image features or non-image features for a portion of the data of the case video data storage 210 input to the first layer 310 (e.g., video data).

The second layer 312 can correspond to the second portion of the environment processing system 220 as discussed herein. The second layer 312 can include a second clip model 322, a second layer processor 332, and a second feature processor 342, and can provide output to a layer output 354. The second clip model 322 can include one or more instructions to receive a video divided into one or more frames and identify one or more timestamps or times of capture associated with those one or more frames. The second layer processor 332 can include a second RNN to identify one or more image features or non-image features as input to the second feature processor 342. The second feature processor 342 can generate one or more of the image features or non-image features for a portion of the data of the case video data storage 210 input to the second layer 312 (e.g., video data).

The third layer 314 can correspond to the third portion of the environment processing system 220 as discussed herein. The third layer 314 can include a third clip model 324, a third layer processor 334, and a third feature processor 344 and can provide output to a layer output 354. The third clip model 324 can include one or more instructions to receive a video divided into one or more frames and to identify one or more timestamps or times of capture associated with those one or more frames. The third layer processor 334 can include a third RNN to identify one or more image features or non-image features as input to the third feature processor 344. The third feature processor 344 can generate one or more of the image features or non-image features for a portion of the data of the case video data storage 210 input to the third layer 314 (e.g., video data).

The fourth layer 316 can correspond to the fourth portion of the environment processing system 220 as discussed herein. The fourth layer 316 can include a fourth clip model 326, a fourth layer processor 336, and a fourth feature processor 346 and can provide output to a layer output 354. The fourth clip model 326 can include one or more instructions to receive a video divided into one or more frames and to identify one or more timestamps or times of capture associated with those one or more frames. The fourth layer processor 336 can include a fourth RNN to identify one or more image features or non-image features as input to the fourth feature processor 346. The fourth feature processor 346 can generate one or more of the image features or non-image features for a portion of the data of the case video data storage 210 input to the fourth layer 316 (e.g., video data).

The mixer 350 can aggregate output from each of the first, second, third, and fourth layers 310, 312, 314, and 316. For example, the mixer 350 can fuse one or more of the image features, the non-image features, or any combination thereof, as discussed herein. Thus, the mixer 350 can provide a fused output 352 based on predictions output by each of the first, second, third, and fourth layers 310, 312, 314, and 316. The layer output 354 can correspond to the output of the first layer 310. For example, the layer output 354 can correspond to a prediction output by the first layer 330. The layer output 354 is not limited to the example illustrated herein. For example, one or more of the second, third, and fourth layers 312, 314, and 316 can provide layer outputs that correspond at least partially in one or more of the structures and operations to the layer output 354.

For example, the one or more physical positions of the one or more body parts each correspond to the respective poses of the one or more body parts engaged with the one or more components of the robotic system or instrument. For example, respective poses can include a slouched position of a surgeon, an upright sitting position of a surgeon, a grip with a straight wrist in line with a manipulator, a grip turned inward with respect to a manipulator, or any combination thereof. Thus, the cameras, as discussed herein, can determine one or more of the positions of one or more of the body parts of a surgeon, including, but not limited to, digits, wrists, arms, forearms, shoulders, upper back, lower back, or any portion thereof, or any combination thereof.

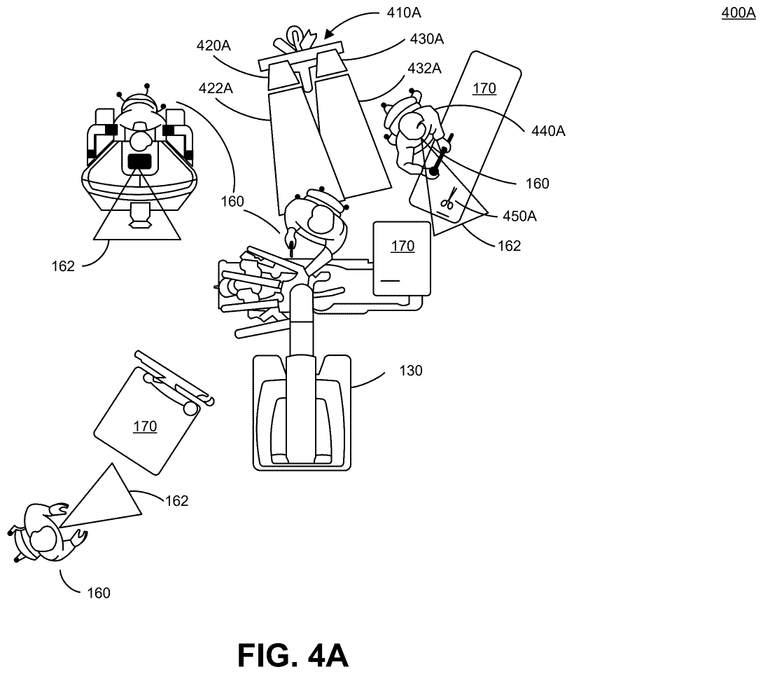

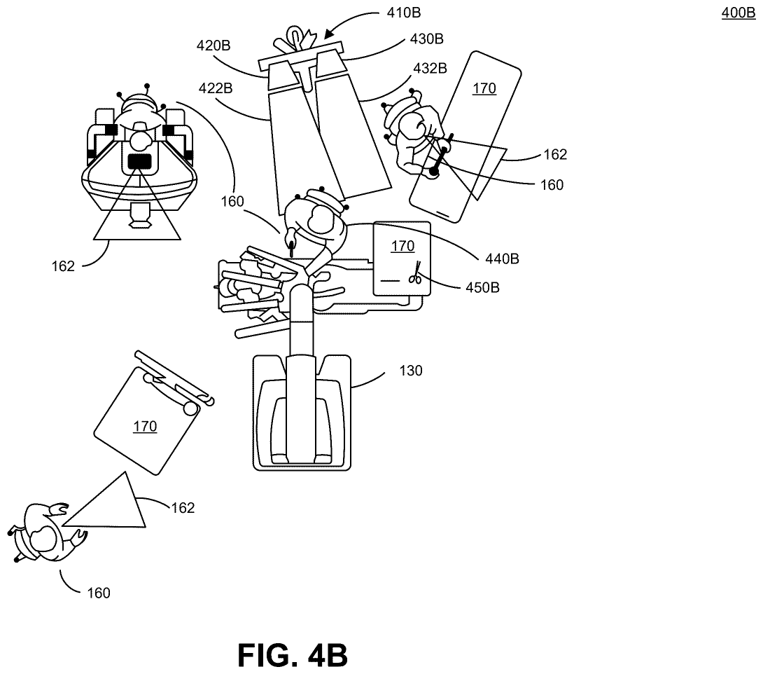

FIG. 4A depicts an example of a first state of a medical environment according to this disclosure. As illustrated by way of example in FIG. 4A, a first state of a medical environment 400A can include at least an exocentric sensor system 410A, an egocentric sensor system 440A, and a medical instrument 450A. The medical instrument 450A can correspond to an object 170 located inside the field of view of the egocentric sensor system 440A at the first time. For example, the medical instrument 450A is a pair of forceps on a first table away from the patient site.

The exocentric sensor system 410A can correspond at least partially in one or more of structure and operation to the sensor system 140 at a first time during a medical procedure. For example, the exocentric sensor system 410A can be positioned on a stand facing toward a patient site, and it can be substantially stationary at the first time during the medical procedure. For example, the exocentric sensor system 410A can be configured to detect the presence of the medical instrument 450A, or it can be configured to provide one or more images or frames of video of the data processing system 110 to detect the presence of the medical instrument 450A. The exocentric sensor system 410A can include a first sensor 420A and a second sensor 430A. The first sensor 420A can correspond at least partially in one or more of the structures and operations to the first camera of the exocentric sensor system 410A. The first sensor 420A can include a field of view 422A. For example, the field of view 422A can correspond to a first stereoscopic view from the exocentric sensor system 410A. For example, the field of view 422A can correspond to a first panoramic view from the exocentric sensor system 410A. The second sensor 430A can correspond at least partially in one or more of structure and operation to a second camera of the exocentric sensor system 410A. The second sensor 430A can include a field of view 432A. For example, the field of view 432A can correspond to a second stereoscopic view from the exocentric sensor system 410A. For example, the field of view 432A can correspond to a second panoramic view from the exocentric sensor system 410A.

Here, the exocentric sensor system 410A or the data processing system 110 can determine absence of the medical instrument 450A at a first given location in the medical environment at the first time, where the first given location corresponds to one or more of the fields of view 422A and 432A. In a stereoscopic mode, the exocentric sensor system 410A, or the data processing system 110, can determine absence of the medical instrument 450A at the first given location at the first time, based on a lack of detection of the medical instrument 450A in both of the fields of view 422A and 432A. In a panoramic mode, the exocentric sensor system 410A or the data processing system 110 can determine absence of the medical instrument 450A at the first given location at the first time, based on a lack of detection of the medical instrument 450A in either of the fields of view 422A and 432A.

The egocentric sensor system 440A can correspond at least partially in one or more of structure and operation to the sensor system 140 at a first time during a medical procedure. For example, the egocentric sensor system 440A can be positioned on a headset of a person 160 and can be substantially mobile at the first time during the medical procedure. For example, the egocentric sensor system 440A can be configured to detect presence of the medical instrument 450A, or can be configured to provide one or more images or frames of video to the data processing system 110 to detect presence of the medical instrument 450A. The egocentric sensor system 440A can include a camera. The camera can include a field of view 442A. The field of view 442A can correspond to an FPV as discussed herein corresponding to the person 160 wearing the headset including the egocentric sensor system 440A. Here, the egocentric sensor system 440A or the data processing system 110 can determine presence of the medical instrument 450A at a second given location in the medical environment at the first time, where the second given location corresponds to the field of view 442A. For example, the egocentric sensor system 440A or the data processing system 110 can determine presence of the medical instrument 450A at the second given location at the first time, based on detecting the medical instrument 450A in the field of view 442A.

FIG. 4B depicts an example of a second state of a medical environment, according to this disclosure. As illustrated by way of example in FIG. 4B, a second state of a medical environment 400B can include at least an exocentric sensor system 410B, an egocentric sensor system 440B, and a medical instrument 450B. The medical instrument 450B can correspond to an object 170 located inside a field of view of the exocentric sensor system 410A at a second time. For example, the medical instrument 450A is a pair of forceps on a second table near the patient site.

The exocentric sensor system 410B can correspond at least partially to one or more of structure and operation to the sensor system 140 at a second time during a medical procedure. For example, the exocentric sensor system 410B can be positioned on a stand facing toward a patient site, and can be substantially stationary at the second time during the medical procedure. For example, the exocentric sensor system 410B can be configured to detect presence of the medical instrument 450B, or can be configured to provide one or more images or frames of video the data processing system 110 to detect presence of the medical instrument 450B. The exocentric sensor system 410B can include a first sensor 420B and a second sensor 430B. Here, the exocentric sensor system 410B or the data processing system 110 can determine presence of the medical instrument 450B at the first given location in the medical environment at the second time, where the first given location corresponds to one or more of the fields of view 422B and 432B. In the stereoscopic mode, the exocentric sensor system 410B or the data processing system 110 can determine presence of the medical instrument 450B at the first given location at the second time, based on detecting the medical instrument 450B in both of the fields of view 422B and 432B. In the panoramic mode, the exocentric sensor system 410B or the data processing system 110 can determine presence of the medical instrument 450B at the first given location at the second time, based on detecting the medical instrument 450B in either of the fields of view 422B or 432B.

The egocentric sensor system 440B can correspond at least partially in one or more of structure and operation to the sensor system 140 at a second time during a medical procedure. For example, the egocentric sensor system 440B can be positioned on a headset of a person 160 and can be substantially mobile at the second time during the medical procedure. For example, the egocentric sensor system 440B can be configured to detect presence of the medical instrument 450B, or can be configured to provide one or more images or frames of video the data processing system 110 to detect presence of the medical instrument 450B. The egocentric sensor system 440B can include a camera of the egocentric sensor system 440B. The camera can include a field of view 442B. The field of view 442B can correspond to an FPV as discussed herein corresponding to the person 160 wearing the headset including the egocentric sensor system 440B. Here, the egocentric sensor system 440B or the data processing system 110 can determine absence of the medical instrument 450B at a second given location in the medical environment at the second time, where the second given location corresponds to the field of view 442B. For example, the egocentric sensor system 440B or the data processing system 110 can determine absence of the medical instrument 450B at the second given location at the second time, based on a lack of detection of the medical instrument 450B in the field of view 442B.

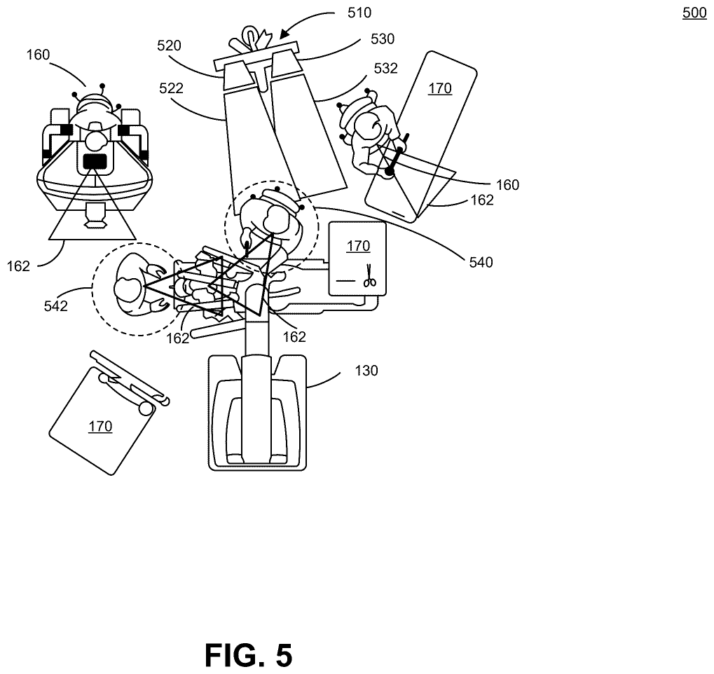

FIG. 5 depicts an example of a medical environment in an interaction state according to this disclosure. As illustrated by way of example in FIG. 5, a medical environment in an interaction state 500 can include at least a sensor system 510, a surgeon 540, and a supervising surgeon 542. The sensor system 510 can correspond at least partially in one or more of structure and operation to the sensor systems 140 or 410A-B. The sensor system 510 can include a first sensor 520, and a second sensor 530. Here, the sensor system 510 can be configured to detect an interaction state based on image data or video data of one or more exocentric sensor system and egocentric sensor systems. For example, the data processing system 110 can determine an interaction by processing one or more image features captured from one or more of the exocentric sensors 520 and 520 associated with the fields of view 522 and 532, and one or more of the egocentric sensors (e.g., wearable or worn headset by the persons 160) associated with one or more of the fields of view 162. The first sensor 520 can correspond at least partially in one or more of structure and operation to the first sensor 420A-B. The first sensor 520 can include a field of view 522. The field of view 522 can correspond at least partially in one or more of structure and operation to the field of view 422A-B. The second sensor 530 can correspond at least partially in one or more of structure and operation to the second sensor 430A-B. The second sensor 530 can include a field of view 532. The field of view 532 can correspond at least partially in one or more of structure and operation to the field of view 432A-B.

The surgeon 540 can be wearing a first egocentric sensor system associated with a first field of view 162. The supervising surgeon 542 can be wearing a second egocentric sensor system associated with a first field of view 162. The data processing system can combine image data, video data, image features, video features, or any combination thereof, based on the fields of view 162, 522 and 532, to identify one or more concurrent interactions in a medical environment (including but not limited to substantially in real-time) at a level of granularity beyond the capability of manual processes. For example, the data processing system can augment a model of the medical environment with image data or image features from the first and second fields of view 162, and can identify an interaction based on the augmented model, the collection of image features from the fields of view 162, 522 and 532, or any combination thereof.

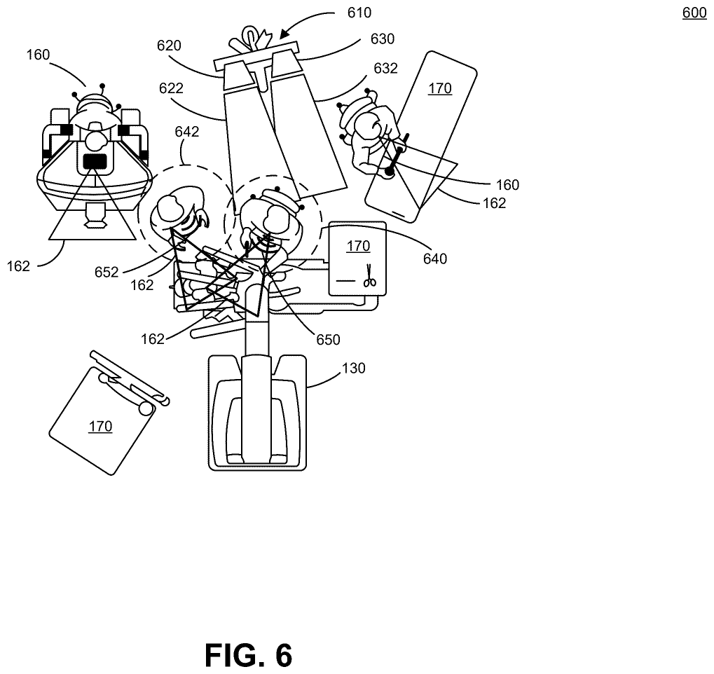

FIG. 6 depicts an example medical environment in a social state according to this disclosure. As illustrated by way of example in FIG. 6, a medical environment in a social state 600 can include at least a sensor system 610, a surgeon 640, a supervising surgeon 642, a surgeon socialization 650, and a supervising surgeon socialization 652. The sensor system 610 can correspond at least partially in one or more of structure and operation to the sensor system 140 or 410A-B. The sensor system 610 can include a first sensor 620, and a second sensor 630. Here, the sensor system 610 can be configured to detect an interaction state based on image data or video data of one or more exocentric sensor system and egocentric sensor systems. For example, the data processing system 110 can determine a interaction by processing one or more image features captured from one or more exocentric sensors 620 and 620 associated with the fields of view 622 and 632, and one or more egocentric sensors (e.g., wearable or worn headset by the persons 160) associated with one or more of the fields of view 162. The first sensor 620 can correspond at least partially in one or more of structure and operation to the first sensor 420A-B. The first sensor 620 can include a field of view 622. The field of view 622 can correspond at least partially in one or more of structure and operation to the field of view 422A-B. The second sensor 630 can correspond at least partially in one or more of structure and operation to the second sensor 430A-B. The second sensor 630 can include a field of view 632. The field of view 632 can correspond at least partially in one or more of structure and operation to the field of view 432A-B.

The surgeon 640 can be wearing a first egocentric sensor system associated with a first field of view 162. For example, the first egocentric sensor system can include a first microphone configured to detect voice or speech from the surgeon 640 or sound near the surgeon 640. The supervising surgeon 642 can be wearing a second egocentric sensor system associated with a first field of view 162. For example, the second egocentric sensor system can include a second microphone configured to detect voice or speech from the supervising surgeon 642, or sound near the supervising surgeon 642. The surgeon socialization 650 can correspond to audio data which are sound waveforms captured by the first microphone. The surgeon socialization 650 can include surgeon instructions, confirmations, observations, or any combination thereof, produced by the surgeon 640, but is not limited thereto. The supervising surgeon socialization 652 can correspond to audio data which are sound waveforms captured by the second microphone. The supervising surgeon socialization 652 can include surgeon instructions, confirmations, observations, or any combination thereof, produced by the supervising surgeon socialization 652, but is not limited thereto.

The data processing system can combine image data, video data, image features, video features, audio data, audio features, or any combination thereof, based on the fields of view 162, 522, and 532, to identify one or more concurrent social states in the medical environment (including but not limited to substantially in real-time) at a level of granularity beyond the capability of manual processes. For example, the data processing system can augment a model of the medical environment with image data, image features, audio data, or audio features from the first and second fields of view 162 and the first and second microphones to identify a social state based on the augmented model, the collection of image features from the fields of view 162, 522, and 532, or any combination thereof.

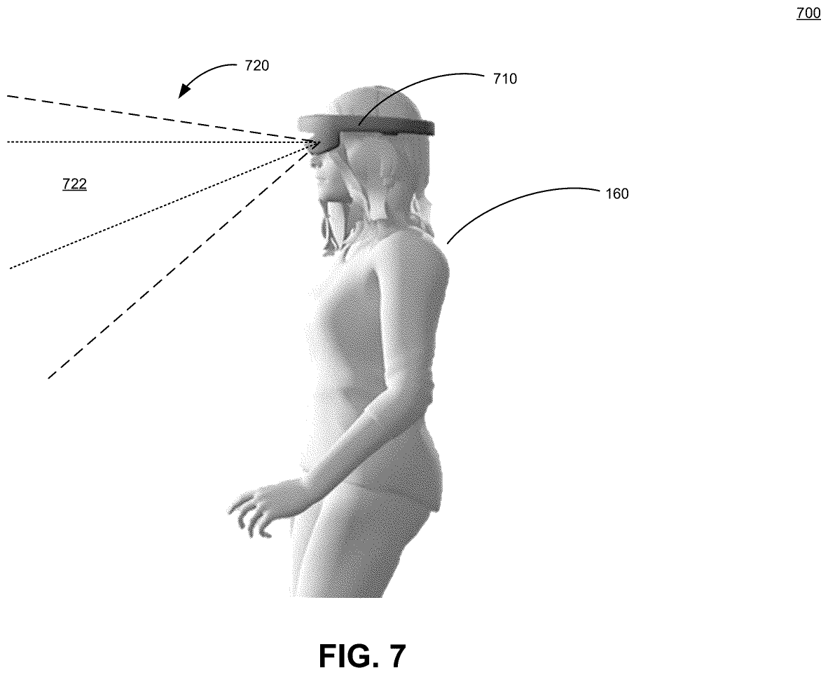

FIG. 7 depicts an example of a first-person view device, according to this disclosure. As illustrated by way of example in FIG. 7, a first-person view device 700 can include at least a headset 710. The headset 710 can include one or more sensors associated with a person 160 as discussed herein. For example, the headset 710 can include a headband and one or more face-mounted or head-mounted electronic devices, including one or more sensors as discussed herein (e.g., cameras and/or microphones). The headset 710 can include a field of view 720. The field of view 720 can correspond at least partially in one or more of structure and operation to an instance of the field of view 162, as discussed herein. The field of view 720 can include a central field of view 722. For example, the central field of view 722 can correspond to an area of focus of the person 160 wearing the headset 710. For example, the data processing system 110 can identify an interaction or a social state based at least partially on one or more image features located in the central field of view 722.

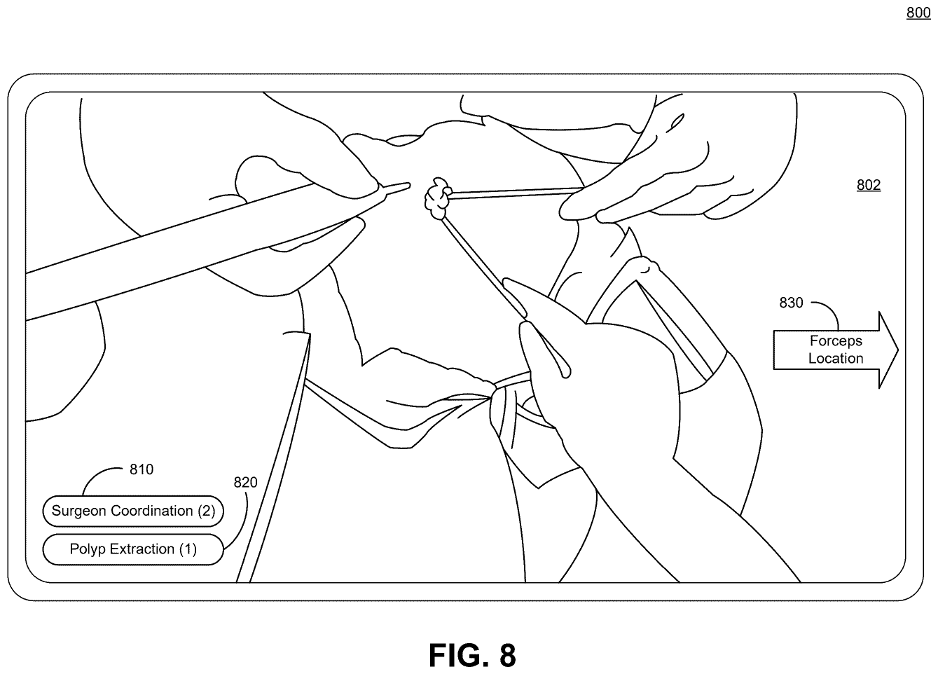

FIG. 8 depicts an example of an augmented reality presentation, according to this disclosure. As illustrated by way of example in FIG. 8, an augmented reality presentation 800 can include at least a field of view 802. For example, the augmented reality presentation 800 can be presented on a display screen integrated with the headset 710, a tablet device, or any combination thereof, but is not limited thereto. The field of view 802 can correspond at least partially to one or more of structure and operation to the field of view 162, 720, or 722. The field of view 802 can include a social state indication 810, an interaction state indication 820, and a memory state indication 830. The presentation 800 is not limited to the content or elements illustrated in FIG. 8 by way of example. For example, a presentation according to this disclosure can include any one or more of the indications 810, 820, and 830.

The social state indication 810 can correspond to a user interface object that indicates or describes a social state determined, as discussed herein (e.g., by the data processing system). For example, the social state indication 810 is a text box or text bubble that includes a social text and a social metric. For example, the social text “Surgeon Coordination” identifies a social state based, for example, in a medical environment, according to FIG. 6. For example, the social metric of “(2)” indicates a number of the person 160 determined by the data processing system 110 for the social state based, for example, in the medical environment according to FIG. 6.

The interaction state indication 820 can correspond to a user interface object that indicates or describes the interaction state determined, as discussed herein (e.g., by the data processing system). For example, the interaction state indication 820 is a text box or text bubble that includes interaction text and an interaction metric. For example, the interaction text “Polyp Extraction” identifies an interaction state based, for example, in a medical environment according to any of the FIGS. 4A-6, or robot metrics, at the first time or second time corresponding to the presentation 800. For example, the interaction metric of “(1)” indicates a number of the persons 160 determined by the data processing system 110 for the interaction state based, for example, on the medical environment according to FIG. 6.

The memory state indication 830 can correspond to a user interface object that indicates or describes a memory state determined as discussed herein (e.g., by the data processing system). For example, the memory state indication 830 is a text box or text bubble that includes memory text and is shaped to indicate a location. For example, the memory text of “Forceps” identifies an object in the medical environment 100B (e.g., forceps 450A or 450B) according to any of FIGS. 4A-B at the first time or the second time corresponding to the presentation 800. For example, the text box is shaped in an arrow to indicate a relative position of the object 450A or 450B relative to the person 160 associated with the presentation 800 (e.g., an arrow pointing right to indicate that the forceps are to the right of the viewer).

In an aspect, one or more of the social state indication 810, the interaction state indication 820, and the memory state indication 830 can be presented with one or more aspects dependent on a role of a viewer or a role of personnel in the medical environment. In an aspect, the social state indication 810, the interaction state indication 820, and the memory state indication 830 can include a level of detail of descriptive text, differing text, or presence or absence of content determined according to an identifier of a person wearing an egocentric sensor, or a type of role associated with the person wearing the egocentric sensor. For example, content intended for a surgeon may be presented only at headsets associated with surgeons, and content intended for a nurse may be presented only at headsets associated with nurses.

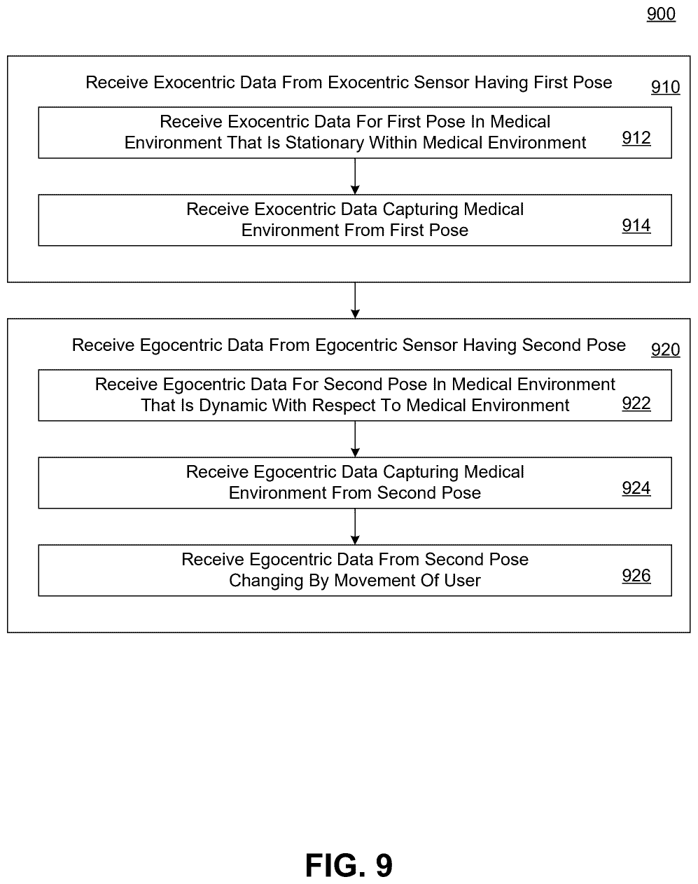

FIG. 9 depicts an example method of identifying procedural states of an environment by fusion of egocentric and exocentric sensor data according to this disclosure. At least the data processing system 110 can perform method 900. In an aspect, the method can include generating, by a machine learning model receiving as input the exocentric data and the egocentric data, a metric indicative of a state of at least one person or object in the medical environment during a portion of a workflow of the medical procedure. In an aspect, the environment processing system 220 can generate, by a machine learning model receiving as input the exocentric data and the egocentric data, a metric indicative of a state of at least one person or object in the medical environment during a portion of a workflow of the medical procedure.

At 910, the method 900 can receive exocentric data from an exocentric sensor having a first pose. At 912, the method 900 can receive exocentric data for the first pose in a medical environment that is stationary within the medical environment. At 914, the method 900 can receive exocentric data capturing the medical environment from the first pose. At 920, the method 900 can receive egocentric data from an egocentric sensor having a second pose. In an aspect, the exocentric data is structured according to a coordinate frame defined relative to the medical environment, and the egocentric data is structured according to the coordinate frame. At 922, the method 900 can receive egocentric data for the second pose in the medical environment that is dynamic with respect to the medical environment. At 924, the method 900 can receive egocentric data capturing the medical environment from the second pose. At 926, the method 900 can receive egocentric data from a second pose changing according to movement of a user.

In an aspect, the method can include receiving event data from a robotic system in the medical environment and the medical procedure. The method can include generating, by the machine learning model receiving as input the event data, the metric. In an aspect, the sensor mode scheduling system 210 can receive event data from a robotic system in the medical environment and the medical procedure. The system can generate, by the machine learning model receiving as input the event data, the metric. In an aspect, the method can include fusing, by a second machine learning model configured to identify one or more features in one or more images, the exocentric data and the egocentric data into the fused data. In an aspect, the environment processing system 220 can fuse, by a second machine learning model configured to identify one or more features in one or more images, the exocentric data and the egocentric data into the fused data.

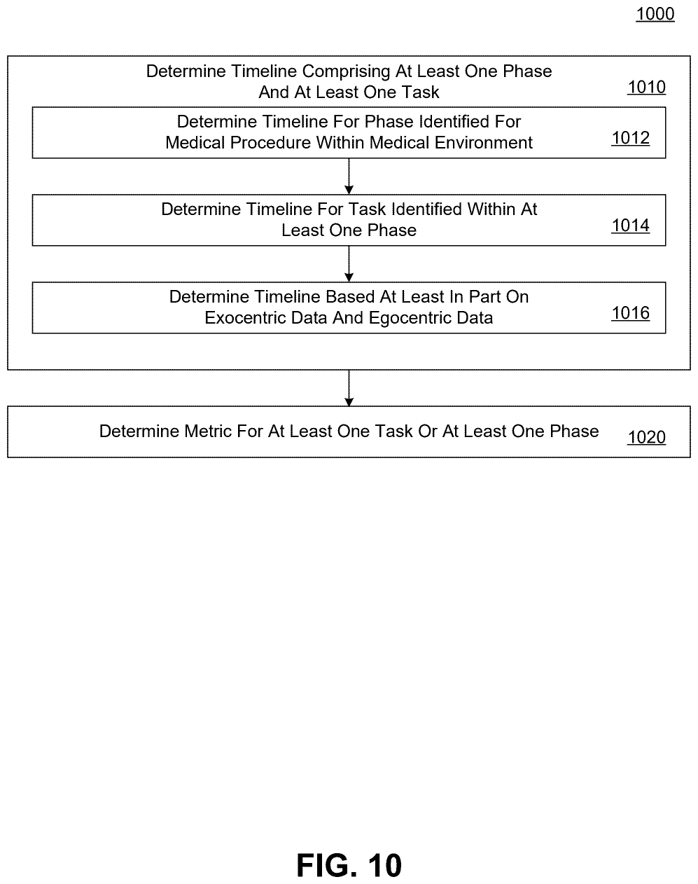

FIG. 10 depicts an example method of identifying procedural states of an environment by fusion of egocentric and exocentric sensor data according to this disclosure. At least the data processing system 110 can perform method 1000.

At 1010, the method 1000 can determine a timeline comprising at least one phase and at least one task. At 1012, the method 1000 can determine the timeline for a phase identified for a medical procedure within the medical environment. At 1014, the method 1000 can determine the timeline for a task identified within the at least one phase. At 1016, the method 1000 can determine the timeline based at least in part on the exocentric data and the egocentric data. In an aspect, the environment processing system 220 can determine the timeline by a machine learning model receiving as input the exocentric data and the egocentric data.

In an aspect, the method can include determining, by the machine learning model receiving as input fused data, a location of an object or a person within the medical environment, the fused data based on the exocentric data and the egocentric data. In an aspect, the environment processing system 220 can determine, by the machine learning model receiving as input fused data, a location of an object or a person within the medical environment, the fused data based on the exocentric data, the egocentric data, and the robotic system data. In some examples, the fused data includes the exocentric data, the egocentric data, and the robotic system data that are time-synchronized. In some examples, the fused data includes the exocentric data and the egocentric data that are spatially registered to a common coordinate frame.

At 1020, the method 1000 can determine a metric for the at least one task or the at least one phase. In an aspect, the metric corresponds to a memory metric indicative of a state of the medical environment or a person or object therein at a time or time period during the medical procedure. In an aspect, the metric corresponds to an interaction metric indicative of a change in the state of an object, and where the interaction metric identifies a person in the medical environment correlated with the change in the state of the object. In an aspect, the metric corresponds to a social metric indicative of an action during the medical procedure, and where the social metric identifies a plurality of persons in the medical environment each correlated with the action during the medical procedure. In an aspect, the metric is based on metadata for at least one of a medical procedure of the plurality of medical procedures, a phase of the plurality of phases, a task of the plurality of tasks, a medical environment (e.g., an OR), a hospital, a robotic system or instrument, or medical staff.

At least one aspect is directed to a system. The system can include one or more processors coupled with memory. The system can receive a first set of data from an exocentric sensor, the exocentric sensor being configured to capture information of a medical environment. The system can receive a second set of data from an egocentric sensor, the egocentric sensor being configured to capture egocentric information from a perspective of a first medical personnel in the medical environment. The system can receive a third set of data from a computer-assisted medical system. The system can generate, using one or more machine-learning models, a set of procedure information for a medical procedure performed in the medical environment based on the first set of data from the exocentric sensor, the second set of data from the egocentric sensor, and the third set of data from the computer-assisted medical system.

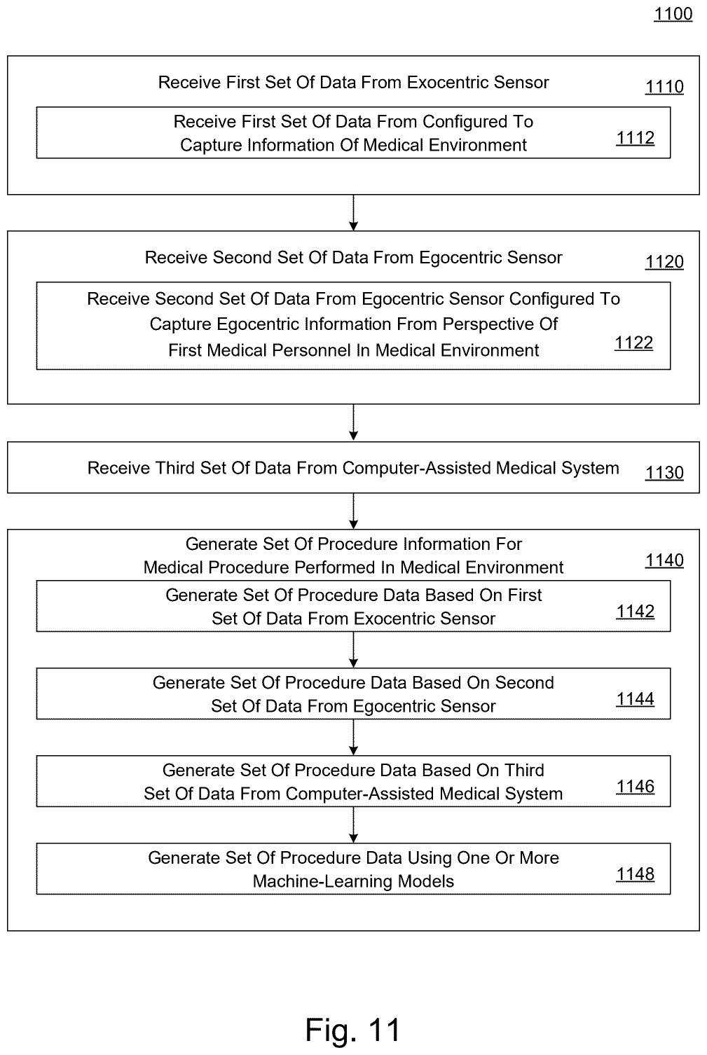

FIG. 11 depicts an example method of monitoring of a medical environment by fusion of egocentric and exocentric sensor data, according to this disclosure. At least the data processing system 110 can perform method 1100.

At least one aspect is directed to a method. The method 1100 can include receiving a first set of data from an exocentric sensor, the exocentric sensor being configured to capture information of a medical environment. The method 1100 can include receiving a second set of data from an egocentric sensor, the egocentric sensor being configured to capture egocentric information from a perspective of a first medical personnel in the medical environment. The method 1100 can include receiving a third set of data from a computer-assisted medical system. The method 1100 can include generating, using one or more machine-learning models, a set of procedure information for a medical procedure performed in the medical environment based on the first set of data from the exocentric sensor, the second set of data from the egocentric sensor, and the third set of data from the computer-assisted medical system.

At 1110, the data processing system 110 can receive a first set of data from an exocentric sensor. For example, the first set of data can include at least one of depth data and color (e.g., RGB) data. In an aspect, the first set of data is structured according to a first coordinate frame defined relative to the medical environment. For example, at 1112, the data processing system 110 can receive the first set of data configured to capture information of a medical environment according to the first coordinate frame (e.g., a first coordinate system). At 1120, the data processing system 110 can receive a second set of data from an egocentric sensor. The second set of data is structured according to a second coordinate frame (e.g., a second coordinate system) for the medical environment. For example, the second set of data can include at least one of depth data and color (e.g., RGB) data. In an aspect, the second set of data includes individual-level information such as a timeline of activities performed by the first medical personnel. The second coordinate frame has an origin or another suitable point of reference that is located on or near the first medical personnel, to correspond to a pose (position and orientation) of the egocentric sensor. For example, at 1122, the data processing system 110 can receive the second set of data from an egocentric sensor configured to capture egocentric information from a perspective of a first medical personnel in the medical environment. In an aspect, the data processing system 110 can generate a set of individual information for the first medical personnel based on the second set of data from the egocentric sensor. At 1130, the data processing system 110 can receive a third set of data from a computer-assisted medical system. For example, the third set of data can include robotic system data, such as at least one of robot event data, or kinematics data as discussed herein, but is not limited thereto.

In some implementations, the data processing system 110 can may receive as input robotic system data and/or data derived from the robotic system data from a robotic manipulator system 130 (e.g., a patient side cart). The robotic system data (or system data) includes kinematics data of a robotic manipulator system, system events data of the robotic manipulator system, input received by the console of the robotic manipulator system from a user, and timestamps associated therewith. The robotic system data of a robotic manipulator system 130 can be generated by the robotic manipulator system 130 in the form of a robotic system log including timestamps in a timeline in its normal course of operations. For example, the kinematics data can indicate configuration(s) of one or more manipulators or manipulator assemblies of the robotic manipulator system 130 as well as the instruments attached or operatively coupled to the manipulators or manipulator assemblies over time throughout the medical procedure.

Furthermore, the system events data can be generated by the robotic manipulator system 130 and can indicate system events of the robotic manipulator system 130. Examples of system events can include, for example, a docking event (e.g., in which manipulator arms are docked to cannulas inserted into a patient anatomy), operator (e.g., surgeon) head-in or head-out event (e.g., indicating a surgeon's head being present or absent at a viewer on a input or control console of the robotic manipulator system), an instrument attachment or removal event (e.g., indicating attachment or removal of an instrument, such as a medical instrument or an imaging instrument, on a manipulator of the robotic manipulator system, a tool exchange event), an instrument change event (e.g., indicating performance of an exchange of one instrument for another instrument for attachment on a manipulator on the robotic manipulator system), a draping-start event or a sterile adapter attachment event (e.g., which may indicate beginning of a sterile draping process), and the like. The robotic manipulator system 130 can include one or more sensors (e.g., camera, infrared sensor, ultrasonic sensors, etc.), actuators, interfaces, consoles, that can output information used to detect such a system event. Such robotic system data can be in natural language (e.g., a string of characters). In other words, the robotic system data can be indicative of one or more states of one or more components of the robotic manipulator system 130. Components of the robotic manipulator system 130 can include, but are not limited to, actuators on the robotic manipulator system 130 and instruments coupled or attached to the actuators of the robotic manipulator system 130. For example, the robotic manipulator system data can include one or more data points indicative of one or more of an activation state (e.g., activated or deactivated), a position, or orientation of a component of the robotic manipulator system 130. For example, the robotic system data can be linked with or correlated with one or more medical procedures, one or more phases of a given medical procedure, one or more tasks of a given phase of a given medical procedure, data from the exocentric sensors, data from the egocentric sensors, and so on. For example, a type of robotic system data can be indicative of a position of one or more actuators of a given arm (or an instrument attached thereto) of the robotic manipulator system 130 at a given time or over a given time interval. The position of an actuator of an arm of the robotic manipulator system 130 can be associated with a given timestamp and correlated with the timestamps of the data from the exocentric and egocentric sensors.

At 1140, the data processing system 110 can generate a set of procedure information for a medical procedure performed in the medical environment. In an aspect, the set of procedure information is indicative of a state of the medical environment at a time or time period during the medical procedure. In an aspect, the set of procedure information is indicative of a change in a state of an object, and identifies a person in the medical environment correlated with the change in the state of the object. In an aspect, the set of procedure information is indicative of an action during the medical procedure, and identifies a plurality of persons in the medical environment each correlated with the action during the medical procedure. At 1142, the data processing system 110 can generate the set of procedure data based on the first set of data from the exocentric sensor. In an aspect, the system can generate, based on the first set of data, the set of procedure information to include a summary of at least a portion of a medical procedure with respect to the medical environment from a perspective of the exocentric sensor. At 1144, the data processing system 110 can generate the set of procedure data based on the second set of data from the egocentric sensor. In an aspect, the system can generate, based on the second set of data, the set of procedure information to include a summary of at least a portion of a medical procedure with respect to the medical environment from the perspective of the first medical personnel. At 1146, the data processing system 110 can generate the set of procedure data based on the third set of data from the computer-assisted medical system.

At 1148, the data processing system 110 can generate the set of procedure data using one or more machine-learning models. In an aspect, the data processing system 110 generate, using a machine learning model receiving as input the first set of data and the second set of data, a state of the first medical personnel in the medical environment during a portion of the medical procedure. In an aspect, the system can generate, by a machine learning model receiving as input the first set of data and the second set of data, a state of the first medical personnel in the medical environment during a portion of the medical procedure. In an aspect, the data processing system 110 generate, by the machine learning model receiving as input the third set of data, the state. In an aspect, the data processing system 110 can generate, using the machine learning model receiving as input the third set of data, the state. In an aspect, the data processing system 110 can determine, using the machine learning model receiving as input fused data, a location of the first medical personnel within the medical environment, the fused data based on the first set of data and the second set of data. In an aspect, the data processing system 110 can determine, using the machine learning model receiving as input fused data, a location of the first medical personnel within the medical environment, the fused data based on the first set of data and the second set of data. In an aspect, the data processing system 110 can include fusing, by a second machine learning model configured to identify one or more features in one or more images, the first set of data and the second set of data into the fused data. In an aspect, the data processing system 110 can fuse, using a second machine learning model configured to identify one or more features in one or more images, the first set of data and the second set of data into the fused data. In some examples, the fused data includes the first, second, and third sets of data that are time-synchronized. In some examples, the fused data includes the first and second sets of data that are spatially registered to a common coordinate frame.

In an aspect, the system can determine, a correspondence between one or more timestamps of the first set of data and the timeline. The system can synchronize, according to the correspondence, the second set of data with the first set of data into the first coordinate frame. In an aspect, the system can determine, according to the correspondence, that at least a portion of an object in the medical environment is occluded from a perspective of the exocentric sensor, and that the portion of the object is at least partially visible from the perspective of the first medical personnel. The system can obtain, responsive to the determination, the second set of data from the egocentric sensor. In an aspect, the system can determine a timeline corresponding to the set of procedure information, by a machine learning model receiving as input the first set of data and the second set of data.

At least one aspect is directed to the data processing system 110. The data processing system 110 can include one or more processors coupled with memory. The data processing system 110 can receive a first set of data from an exocentric sensor, the exocentric sensor being configured to capture information of a medical environment. The data processing system 110 can receive a second set of data from an egocentric sensor, the egocentric sensor being configured to capture egocentric information from a perspective of a first medical personnel in the medical environment. The data processing system 110 can receive a third set of data from a computer-assisted medical system. The system can generate, using one or more machine-learning models, a set of procedure information for a medical procedure performed in the medical environment based on the first set of data from the exocentric sensor, the second set of data from the egocentric sensor, and the third set of data from the computer-assisted medical system.

In some embodiments, the first set of data obtained from at least one exocentric sensor, the second set of data obtained from at least one egocentric sensor, and the third set of data (e.g., robotic system data) can be time-synchronized and integrated. In some examples, a system clock of an exocentric sensor, a system clock of an egocentric sensor, and the system block of the robotic manipulator system 130 are synchronized to a reference clock, such as the clock of the exocentric sensor, the clock of the egocentric sensor, the block of the robotic manipulator system 130, the clock of the data processing system 110, the clock of the sensor mode scheduling system 210, the clock of the environment processing system 220, or another suitable third party clock, such that the timestamps of the first, second, and third sets of data are already synchronized as the first, second, and third sets of data are collected.

In some examples, time-synchronization can include aligning one or more timestamps of the first set of data, one or more timestamps of the second set of data, and one or more timestamps of the third set of data, according to detection of an object, person, and so on. As described herein, the first and second sets of data can include depth and/or visual videos with timestamps, which may not have been aligned. By performing object detection and identification in the manner described herein, a same object or person may be detected and identified using both the first and second sets of data. In some cases, timestamps of the first and second sets of data at which a same object or person is detected and/or identified can be aligned. For example, timestamps of the first and second sets of data at which forceps is first detected and/or identified can be aligned. In some cases, timestamps of the first and second sets of data at which a same object or person is detected and/or identified at a same position can be aligned. For example, timestamps of the first and second sets of data at which forceps is first detected and/or identified at a same set of coordinates (e.g., within a predetermined distance threshold) within the common coordinate frame can be aligned.

In some cases, timestamps of the first, second, and third sets of data at which a same action, task, operation, phase begins or ends can be aligned. For example, timestamps of the first, second, and third sets of data marking the beginning or end of a tool-on task in which an instrument is attached to a robotic manipulator system 130 can be aligned. An action, task, operation, phase, can be detected according to a location of an object or a person, a gesture or motion of a person, a movement pattern of a person or object, and so on, using the system 300. The robotic system data of the third set of data can output timestamps of system events and kinematic events as described, and an example of the system events include a tool-on event and its associated timestamp.

Spatial registration can correspond to transforming the coordinate frames or systems of the first and second sets of data to a common coordinate frame or system for determining locations, positions, and movements of objects and persons within the medical environment. In some embodiments, exocentric sensors are located in a fixed location in the medical environment and have a specific field of view in a first coordinate frame or system. In some embodiments, sensors placed or worn on or near a body (e.g., a head, a chest, a hand, etc.) of a person has a specific field of view in a second coordinate frame or system. In other words, a 3D volume corresponding to the medical environment in which exocentric and egocentric sensors are located can be expressed in both the first and second coordinate frames or systems. Spatial registration can transform information in the first and/or second coordinate frames to information in a common coordinate frame. In some examples, the common coordinate frame can be the first coordinate frame, thus the second set of data can be transformed from the second coordinate frame to the first coordinate frame. For example, a pose (e.g., a position or orientation) of an egocentric sensor can be detected via object detection and identification of the system 300 in the manner described herein, where the pose of the egocentric sensor is defined using coordinates in the first coordinate frame. Using suitable transformation methods such as 3D transformation matrices according to the known pose of the egocentric sensor in the first coordinate frame, the second set of data can be projected to the first coordinate frame.

At least one aspect is directed to a non-transitory computer readable medium can include one or more instructions stored thereon and executable by a processor 112 of the data processing system 110. The processor 112 can receive a first set of data from an exocentric sensor, the exocentric sensor being configured to capture information of a medical environment. The processor 112 can receive a second set of data from an egocentric sensor, the egocentric sensor being configured to capture egocentric information from a perspective of a first medical personnel in the medical environment. The processor 112 can receive a third set of data from a computer-assisted medical system. The processor 112 can generate, using one or more machine-learning models, a set of procedure information for a medical procedure performed in the medical environment based on the first set of data from the exocentric sensor, the second set of data from the egocentric sensor, and the third set of data from the computer-assisted medical system. For example, the system memory 114 can correspond to the non-transitory computer readable medium. In an aspect, a non-transitory computer readable medium can include one or more instructions executable by the processor 112. The processor 112 can generate, via a machine learning model receiving as input the first set of data and the second set of data, a state of the first medical personnel in the medical environment during a portion of the medical procedure.

In an aspect, the environment processing system 220 can generate, based on the exocentric data, procedure-level data that corresponds to a summary of at least a portion of the medical procedure with respect to the medical environment from the first pose. For example, procedure-level data can correspond to data indicative of a state of the medical environment, the medical environment or the medical procedure, and is not limited to any pf the persons 160. For example, procedure-level data can correspond to text indicative of “The patient preparation was performed by three nurses and an anesthesiologist in above-average time.” In an aspect, the environment processing system 220 can generate, based on the egocentric data, individual-level data that corresponds to a summary of at least a portion of the medical procedure with respect to the medical environment from the second pose. For example, individual-level data can correspond to data indicative of a state of one of the persons 160. For example, individual-level data can correspond to text indicative of “The supervising surgeon intervened twice during the polyp removal phase.”

Having now described some illustrative implementations, the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both “A’ and ‘B’. Such references used in conjunction with “comprising” or other open terminology can include additional items. References to “is” or “are” may be construed as nonlimiting to the implementation or action referenced in connection with that term. The terms “is” or “are” or any tense or derivative thereof, are interchangeable and synonymous with “can be” as used herein, unless stated otherwise herein.

Directional indicators depicted herein are example directions to facilitate understanding of the examples discussed herein, and are not limited to the directional indicators depicted herein. Any directional indicator depicted herein can be modified to the reverse direction, or can be modified to include both the depicted direction and a direction reverse to the depicted direction, unless stated otherwise herein. While operations are depicted in the drawings in a particular order, such operations are not required to be performed in the particular order shown or in sequential order, and all illustrated operations are not required to be performed. Actions described herein can be performed in a different order. Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any clam elements.

Scope of the systems and methods described herein is thus indicated by the appended claims, rather than the foregoing description. The scope of the claims includes equivalents to the meaning and scope of the appended claims.

What is claimed is:

1. A system, comprising:
one or more processors coupled with memory to:
receive a first set of data from an exocentric sensor, the exocentric sensor being configured to capture information of a medical environment;
receive a second set of data from an egocentric sensor, the egocentric sensor being configured to capture egocentric information from a perspective of a first medical personnel in the medical environment;
receive a third set of data from a computer-assisted medical system; and
generate, using one or more machine-learning models, a set of procedure information for a medical procedure performed in the medical environment based on the first set of data from the exocentric sensor, the second set of data from the egocentric sensor, and the third set of data from the computer-assisted medical system.

2. The system of claim 1, wherein the one or more processors further generate a set of individual information for the first medical personnel based on the second set of data from the egocentric sensor.

3. The system of claim 2, wherein the second set of data is individual-level information that includes a timeline of activities performed by the first medical personnel.

4. The system of claim 1, the processors to:
generate, by a machine learning model receiving as input the first set of data and the second set of data, a state of the first medical personnel in the medical environment during a portion of the medical procedure.

5. The system of claim 4, the processors to:
generate, by the machine learning model receiving as input the third set of data, the state.

6. The system of claim 4, the processors to:
determine, by the machine learning model receiving as input fused data, a location of the first medical personnel within the medical environment, the fused data based on the first set of data and the second set of data.

7. The system of claim 4, the processors to:
fuse, by a second machine learning model configured to identify one or more features in one or more images, the first set of data and the second set of data into the fused data.

8. The system of claim 1, wherein the first set of data is structured according to a first coordinate frame defined relative to the medical environment, and the second set of data is structured according to a second coordinate frame for the medical environment.

9. The system of claim 8, the processors to:
temporally synchronize the first set of data with the second set of data; and
spatially register the second set of data with the first coordinate frame.

10. The system of claim 8, the processors to:
determine, according to the correspondence, that at least a portion of an object in the medical environment is occluded from a perspective of the exocentric sensor, and that the portion of the object is at least partially visible from the perspective of the first medical personnel; and
obtain, responsive to the determination, the second set of data from the egocentric sensor.

11. The system of claim 1, wherein the set of procedure information is indicative of a state of the medical environment at a time or time period during the medical procedure.

12. The system of claim 1, wherein the set of procedure information is indicative of a change in a state of an object, and identifies a person in the medical environment correlated with the change in the state of the object.

13. The system of claim 1, wherein the set of procedure information is indicative of an action during the medical procedure, and identifies a plurality of persons in the medical environment each correlated with the action during the medical procedure.

14. The system of claim 1, the processors to:
determine a timeline corresponding to the set of procedure information, by a machine learning model receiving as input the first set of data and the second set of data.

15. The system of claim 1, wherein the set of procedure information is based on metadata for at least one of:
one or more medical procedures corresponding to the medical environment;
a phase of a plurality of phases of the one or more medical procedures;
a task of a plurality of tasks of the plurality of phases;
an operating room (OR);
a hospital;
a robotic system or instrument; or
medical personnel.

16. The system of claim 1, the processors to:
generate, based on the first set of data, the set of procedure information including a summary of at least a portion of a medical procedure with respect to the medical environment from a perspective of the exocentric sensor; and
generate, based on the second set of data, the set of procedure information including a summary of at least a portion of a medical procedure with respect to the medical environment from the perspective of the first medical personnel.

17. A method, comprising:
receive a first set of data from an exocentric sensor, the exocentric sensor being configured to capture information of a medical environment;
receive a second set of data from an egocentric sensor, the egocentric sensor being configured to capture egocentric information from a perspective of a first medical personnel in the medical environment;
receive a third set of data from a computer-assisted medical system; and
generate, using one or more machine-learning models, a set of procedure information for a medical procedure performed in the medical environment based on the first set of data from the exocentric sensor, the second set of data from the egocentric sensor, and the third set of data from the computer-assisted medical system.

18. The method of claim 17, further comprising:
generating, by a machine learning model receiving as input the first set of data and the second set of data, a state of the first medical personnel in the medical environment during a portion of the medical procedure;
generating, by the machine learning model receiving as input the third set of data, the state;
determining, by the machine learning model receiving as input fused data, a location of the first medical personnel within the medical environment, the fused data based on the first set of data and the second set of data; and
fusing, by a second machine learning model configured to identify one or more features in one or more images, the first set of data and the second set of data into the fused data.

19. A non-transitory computer readable medium including one or more instructions stored thereon and executable by a processor to:
receive a first set of data from an exocentric sensor, the exocentric sensor being configured to capture information of a medical environment, the first set of data including depth data and RGB data;
receive a second set of data from an egocentric sensor, the egocentric sensor being configured to capture egocentric information from a perspective of a first medical personnel in the medical environment, the second set of data including depth data and RGB data;
receive a third set of data from a computer-assisted medical system, the third set of data including robot event data and kinematics data of the computer-assisted medical system; and
generate, using one or more machine-learning models, a set of procedure information for a medical procedure performed in the medical environment based on the first set of data from the exocentric sensor, the second set of data from the egocentric sensor, and the third set of data from the computer-assisted medical system.

20. The non-transitory computer readable medium of claim 19, further including one or more instructions executable by the processor to:
generate, by the processor via a machine learning model receiving as input the first set of data and the second set of data, a state of the first medical personnel in the medical environment during a portion of the medical procedure.
